(19) United States
(12) Patent Application Publication
(10) Pub. No.: US 2005/0153985 A1
(43) Pub. Date: Jul. 14, 2005

(54) Methods of treating acute inflammation in animals with p38 map kinase inhibitors

(76) Inventors: Marcus E. Kehrli JR., Ames, IA (US); Subas M. Sakya, East Lyme, CT (US)

Correspondence Address: PHARMACIA & UPJOHN, 301 HENRIETTA ST, 0228-32-LAW, KALAMAZOO, MI, 49007, US

(21) Appl. No.: 11/014,392

(22) Filed: Dec. 16, 2004

Related U.S. Application Data
(60) Provisional application No. 60/530,722, filed on Dec. 18, 2003.

(51) Int. Cl. A61K 31/497; A61K 31/4745; A61K 31/4192; A61K 31/4439

(52) U.S. Cl. 514/255.05; 514/303; 514/359; 514/341; 514/394

(57) ABSTRACT

The present invention provides methods for treating animals having acute inflammatory conditions, including mastitis, by administering at least one p38 MAP kinase inhibitor. The present invention also provides methods for enhancing milk production and reducing milk discard in animals afflicted with acute inflammatory conditions by administering at least one, p38 MAP kinase inhibitor.

### CROSS-REFERENCE TO RELATED APPLICATIONS

This application claims the benefit of U.S. Provisional Application Ser. No. 60/530,722, filed Dec. 18, 2003 which is incorporated by reference.

### FIELD OF THE INVENTION

The present invention relates to the use of p38 MAP kinase inhibitors for the treatment of animals having acute inflammation and conditions caused thereby. In particular, the present invention provides methods for treating animals having acute inflammatory conditions, including mastitis, by administering at least one p38 MAP kinase inhibitor. The present invention also provides methods for enhancing milk production and reducing milk discard in animals afflicted with acute inflammatory conditions by administering at least one p38 MAP kinase inhibitor.

### BACKGROUND OF THE INVENTION

Acute inflammatory responses in mammals often damage tissue resulting in loss of organ or tissue function and concomitant negative impacts on overall health, production and performance.

Mitogen-activated protein (MAP) kinases are key enzymes involved in signal transduction and the amplification of cellular responses to stimuli. The p38 group of MAP kinases is a group of MAP kinases associated with the onset and progression of inflammation. The p38 group has at least three known homologues of the original p38 MAP kinase (Ono et al. (2000) Cellular Signalling 12:1-13.). Early inflammatory events include cytokine release, activation and rapid accumulation of neutrophils with subsequent recruitment of mononuclear cells. p38 MAP kinase plays a central role in regulating a wide range of inflammatory responses in many different cells. Recent studies have shown that a p38 MAP kinase inhibitor [(S)-5-[2-(1-phenylethylamino)pyrimidin-4-yl]-1-methyl-4-(3-trifluoromethylphenyl)-2-(4-piperidiny) imidazole] reduced initial neutrophil recruitment to the lung in a murine model of mild pulmonary inflammation induced by lipopolysaccharide (LPS) (Nick et al. (2000) J. Immunol. 164:2151-2159.) p38 MAP kinase is activated by dual phosphorylation after stimulation by a wide array of extracellular stimuli including physiochemical stress, treatment with lipopolysaccharide (LPS) or E. coli, signal transduction from the Toll-like receptors, as well as, TNF and IL-1 receptors. The products of the p38 phosphorylation mediate the production of inflammatory cytokines, including TNF, IL-1, IL-6, iNOS and cyclooxygenase-2.

Mastitis is an affliction of lactating dairy cows and is one of the most costly diseases to animal agriculture, with economic losses exceeding $2 billion annually in the United States alone. (Blosser, T. (1979) J. Dairy Sci. 62:119-127). Mastitis is caused by intramammary infection with many bacterial pathogens, including staphylococci, streptococci, and coliforms. Economic losses attributable to mastitis include reduced milk production and quality, increased veterinary costs and death of cows. Reduced milk production is widely attributed to pathophysiologic changes associated with the inflammatory response to bacterial infection. Shuster, D. and Kehrli, M. E. (1995) Am. J. Vet. Res. 56(3):212-320, incorporated herein by reference; Shuster et al. (1991) J. Dairy Sci. 74:1527-1538; Shuster et al. (1991) J. Dairy Sci. 74:3407-3411.; Shuster et al. (1991) J. Dairy Sci. 74:3763-3774. Mastitis caused by the bacteria characterized above can manifest as either clinical or subclinical disease. Cullor et al. (1990) Disorders of the mammary gland in large animal medicine., B. P. Smith, The C.V. Mosby Company, St. Louis. Mo. 63146, pp.1047-1067. Clinical disease varies from mildly affected quarters with changes in the milk, through severely infected quarters with eventual loss of that quarter, to systemically ill cows that often die. Subclinical mastitis is prevalent in many dairy herds. Affected quarters are infected with the pathogenic bacteria described above, but clinical signs are absent. The level of somatic cells increases in the milk, which change can be detected by conventional means. Subclinical mastitis is accompanied by lowered milk production and milk quality.

Here we disclose inhibitors of the kinase activity of p38 useful in a method to treat acute inflammatory conditions characterized by enhanced p38 MAP kinase activity resulting in animals having increased milk production with reduced loss or discard.

### SUMMARY OF THE INVENTION

The present invention provides a method of treating an inflammatory disease or enhancing the recovery from acute inflammatory disease in an animal in need thereof which comprises administering to said animal an effective amount of at least one p38 MAP kinase inhibitor.

Another aspect of the present invention is a method for the enhancement of milk production or reduction of milk loss in an animal suffering from an acute inflammatory disease which comprises the administration to said mammal of an effective amount of at least one p38 MAP kinase inhibitor.

A third aspect of the present invention is directed to a method of inhibiting the synthesis and activity of the COX-2 enzyme, TNF or IL-1 in an animal comprising the administration of an effective amount of at least one p38 MAP kinase inhibitor.

In another aspect the present invention is directed to a method of inhibiting apoptotic cell death in an animal comprising the administration of an effective amount of at least one p38 MAP kinase inhibitor.

In a preferred embodiment, the p38 MAP kinase inhibitor is selected from

(i) the compound of Formula I,

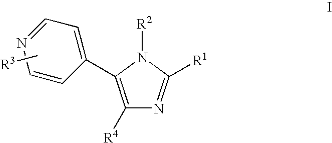

wherein R¹ is —H;

R² is substituted and unsubstituted heterocyclic, cycloalkyl, aryl, heteroaryl: wherein heterocyclic is a 5-, 6- or 7-membered saturated, partially saturated or unsaturated ring containing from one to three heteroatoms independently selected from the group consisting of nitrogen, oxygen and sulfur; and including any bicyclic group in which any of the above heterocyclic rings is fused to a benzene ring or another heterocycle; and the nitrogen may be in the oxidized state giving the N-oxide form; and optionally substituted with R_{y};

R_{y} for each occurrence is independently -halo, —OH, —(C₁-C₆)alkyl, —(C₂-C₆)alkenyl, —(C₂-C₆)alkynyl, —O(C₁-C₆)alkyl, —O(C₂-C₆)alkenyl, —O(C₂-C₆)alkynyl, —(C₀-C₆)alkyl-NR¹³R¹⁴, —C(O)—NR¹³R¹⁴, —SO₂R¹³, —SOR¹³, —SR¹³, —NR¹³—SO₂R¹⁴, —NR¹³—C(O)—R¹⁴, —NR¹³—OR¹⁴, —SO₂—NR¹³R¹⁴, —CN, —CF₃, —C(O)(C₁-C₆)alkyl, ═O, —SO₂-phenyl, or C(O)—Ar or het-Ar;

R³ is independently —H, -halo, —OH, —(C₁-C₁₀)alkyl, OCH₃, NH₂, NHR, wherein R is aryl, heteroaryl or alkyl; and

R⁴ is substituted and unsubstituted aryl and heteroaryl;

R¹³ and R¹⁴ for each occurrence are each independently —H, —(C₁-C₆)alkyl, wherein 1 or 2 carbon atoms, other than the connecting carbon atom, may optionally be replaced with 1 or 2 heteroatoms independently selected from S, O and N and wherein each carbon atom is optionally substituted with 1, 2 or 3 halo; —(C₂-C₆)alkenyl, optionally substituted with 1, 2 or 3 halo; or —(C₂-C₆)alkynyl wherein one carbon atom, other than the connecting carbon atom and the ethynyl atoms, may optionally be replaced with one oxygen atom and wherein each carbon atom is optionally substituted with 1, 2 or 3 halo;

or R¹³ and R¹⁴ are taken together with N to which they are attached to form het;

(ii) the compound of Formula II,

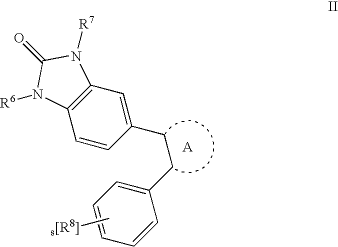

wherein “A” is substituted or unsubstituted pyrrolyl, imidazolyl, pyrazolyl, oxazolyl, isoxazolyl, thiazolyl, or isothiazolyl;

R⁶ and R⁷ are independently H or substituted or unsubstituted (cyclo)alkyl, phenyl, heteroaryl, or heterocyclyl;

R⁸ is independently halo, (perhalo)alkyl, (perhalo)cycloalkyl, alkenyl, alkynyl, heterocyclyl(oxy), phenyl, OH, (perhalo)alkoxy, phenoxy, alkylthio, alkyl(amino)sulfonyl, alkylsulfamoyl, carbamoyl, acyl or carboxy; and

s is 0-5;

(iii) the compound of Formula III

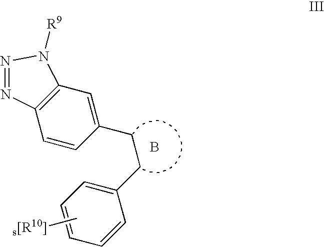

wherein “B” is a substituted or unsubstituted hetero group, such as pyrrolyl, imidazolyl, pyrazolyl, oxazolyl;

R⁹ is H, alkyl, cycloalkyl, heterocycloalkyl, aryl or heteroaryl;

R¹⁰ is H, alkyl, phenyl, F, Cl or CN; and

s is 0-5; or

(iv) the compound of Formula IV,

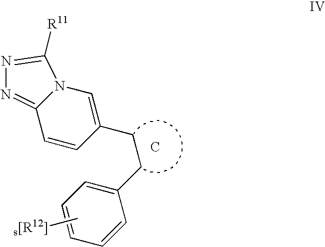

wherein “C” is substituted or unsubstituted pyrrolyl, imidazolyl, pyrazolyl, oxazolyl, isoxazolyl, thiazolyl, or isothiazolyl;

R¹¹ is H, alkenyl, alkynyl, or substituted or unsubstituted (cyclo)alkyl, phenyl, heteroaryl, or heterocyclyl, or amino;

R¹² is halo, (cyclo)alkyl(oxy), (perhalo)alkyl, alkenyl, alkynyl, phenyl, heteroaryl(oxy), heterocyclyl(oxy), OH, (perhalo)alkoxy, phenoxy, alkylthio, alkylsulfonyl, alkylaminosulfonyl, NO₂, substituted and unsubstituted amino or carbamoyl; and

s is 0-5.

In a preferred embodiment, the p38 MAP kinase inhibitor is

(i) a compound MAPKi #1

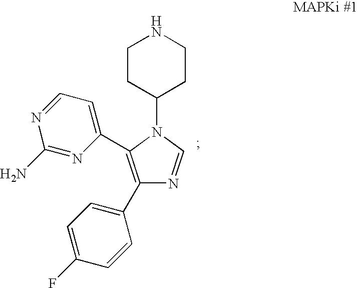

(ii) a compound MAPKi #2

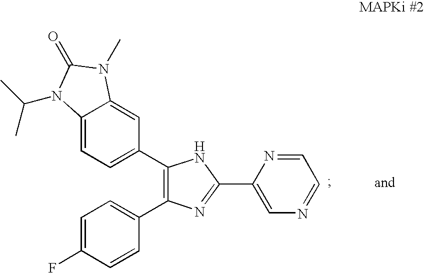

(iii) a compound MAPKi #3

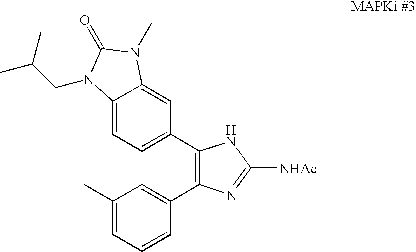

(iv) a compound of Formula IIIa

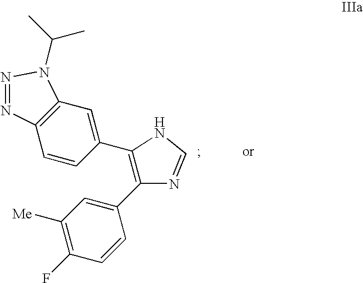

(v) a compound of Formula IVa

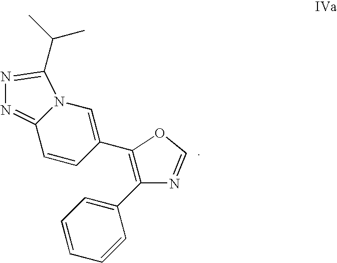

In a preferred embodiment, the inflammatory disease is selected from the group consisting of mastitis, respiratory disease, replaced placenta membranes, metritis, pyometra, enteritis, hepatitis, nephritis, septicemia, endotoxemia, laminitis, frostbite and obstructive bowel problems.

Preferred obstructive bowel problems are selected from the group consisting of colic, displaced abomasums, and cecal torsion.

In a preferred embodiment, the inflammatory disease is mastitis and the animal is a cow.

In a further embodiment, a pharmaceutically acceptable carrier is preferred.

The term “A,” “B,” “C,” “het” or “heterocycle” refers to an optionally substituted hetero group containing one to two heteroatoms selected from nitrogen, sulfur and oxygen, such as pyrrolyl, imidazolyl, pyrazolyl, oxazolyl, isoxazolyl, thiazolyl, or isothiazolyl.

The term “alkyl,” as well as the alkyl moieties of other groups referred to herein (e.g. alkoxy), may be liner or branched (such as methyl, ethyl, n-propyl, isopropyl, n-butyl, iso-butyl, secondary-butyl, tertiary butyl), and they may also be cylic (e.g. cyclopropyl or cyclobutyl).

The term “halogen” includes fluoro, chloro, bromo or iodo or fluoride, chloride, bromide or iodide.

The term “aryl” means aromatic radicals such as phenyl, naphthyl, tetrahydronaphthyl, indanyl and the like, optionally substituted by 1-3 suitable substituents such as fluoro, chloro, trifluoromethyl, (C₁-C₆)alkoxy, (C₆-C₁₀)aryloxy, trifluoromethoxy, difluoromethoxy or (C₁-C₆)alkyl.

The term “heteroaryl” refers to an aromatic heterocyclic group usually with one heteroatom selected from O, S and N in the ring.

The term “heterocyclic” as used herein refers to a cyclic group containing 1-9 carbon atoms and 1-4 hetero atoms selected from N, O, S and NR. Examples of such rings include, inter alia, azetidinyl, terahydrofuranyl, imidazolidinyl, pyrrolidinyl, piperidinyl.

Further examples of the above terms are described more fully in the references cited herein that further describe the compounds utilized in the claimed invention.

The term “treating or treat” with respect to an acute inflammatory condition as used herein means to inhibit, reduce, prevent or ameliorate symptoms associated with inflammatory responses mediated by p38 MAP kinase including the inhibition of Tumor Necrosis Factor (TNF), Interleukin-1 (IL-1) and cycloogygenase-2 (COX-2), and to alleviate the symptoms of inflammatory conditions or diseases caused by amplification of inflammatory cytokines including TNF, IL-1 and COX-2. The treatment is considered therapeutic if there is an enhanced recovery from symptoms of acute inflammation.

An “enhanced recovery” as contemplated by the present invention is conventionally determined from a comparison of the condition of infected, treated animals with infected-non-medicated animals. An enhanced recovery is assessed by any one of the following: an approximate return to the antecedent physiological performance level of the inflamed tissue, such as respiratory function, growth rate, reproductive performance, locomotion, milk synthesis and secretion. Examples might include a reduction in milk discard, increase in milk yield, decrease in inflammation, decreased E. coli levels in milk, or decreased levels of whey PGE₂ levels, for example. The method of the present invention is, for example, effective in enhancing the recovery from acute inflammatory responses in animals.

The term “acute inflammatory condition” as used herein means an affliction or disease of an animal including but not limited to mastitis, respiratory disease, retained placental membranes, metritis, pyometra, enteritis, hepatitis, nephritis, septicemia, laminitis, frostbite and obstructive bowel problems including, colic, displaced abomasums, cecal torsion and endotoxemia.

The term “animal” as used herein refers to all mammals, including but not limited to equids, companion animals and livestock.

The term “cattle” as used herein refers to bovine animals including but not limited to steer, bulls, cows, and calves. Preferably, the method of the present invention is applied to an animal which is a lactating non-human mammal; most preferably, a cow.

The term “effective amount” refers to an amount of at least one p38 MAP kinase inhibitor sufficient to increase milk production, decrease milk discard, decrease E. coli count, or decrease whey PGE₂ levels in animals to which the p38 MAP kinase inhibitor is administered. An effective amount of p38 MAP kinase inhibitor means, for example, that the inhibitor enhances the recovery of an animal afflicted with an acute inflammatory condition or disease.

The term “pharmaceutically acceptable carrier” refers to a carrier medium that does not interfere with the effectiveness of the biological activity of the active ingredient, is chemically inert and is not toxic to the subject to whom it is administered.

### BRIEF DESCRIPTION OF THE DRAWINGS

FIG. 1 depicts the average body temperature of cows administered saline or 30 cfu E. coli into a single quarter followed 13 hours later by administration of MAPKi #1, MAPKi #2, MAPKi #3, or vehicle.

FIG. 2 depicts the average daily milk production of cows administered saline or 30 cfu E. coli into a single quarter followed 13 hours later by administration of MAPKi #1, MAPKi #2, MAPKi #3, or vehicle.

FIG. 3 depicts the average milk clinical scores of cows administered saline or 30 cfu E. coli into a single quarter followed 13 hours later by administration of MAPKi #1, MAPKi #2 MAPKi #3, or vehicle.

FIG. 4 depicts the average gland clinical scores of cows administered saline or 30 cfu E. coli into a single quarter followed 13 hours later by administration of MAPKi #1, MAPKi #2, MAPKi #3, or vehicle.

FIG. 5 depicts the average cumulative clinical score of cows administered saline or 30 cfu E. coli into a single quarter followed 13 hours later by administration of MAPKi #1, MAPKi #2, MAPKi #3, or vehicle.

FIG. 6 depicts the average log 10 of milk somatic cell count (SCC) of cows administered saline or 30 cfu E. coli into a single quarter followed 13 hours later by administration of MAPKi #1, MAPKi #2, MAPKi #3, or vehicle.

FIG. 7 depicts the average total WBC (peripheral blood) of cows administered saline or 30 cfu E. coli into a single quarter followed 13 hours later by administration of MAPKi #1, MAPKi #2, MAPKi #3, or vehicle.

FIG. 8 depicts the average total PMN (peripheral blood) of cows administered saline or 30 cfu E. coli into a single quarter followed 13 hours later by administration of MAPKi #1, MAPKi #2, MAPKi #3, or vehicle.

FIG. 9 depicts the average whey PGE₂ concentration of cows administered saline or 30 cfu E. coli into a single quarter followed 13 hours later by administration of MAPKi #1, MAPKi #2, MAPKi #3, or vehicle.

FIG. 10 depicts bacteria (E. coli) numbers (in ml) from cows administered saline or 30 cfu E. coli into a single quarter followed 13 hours later by administration of MAPKi #1, MAPKi #2, MAPKi #3, or vehicle.

### DETAILED DESCRIPTION

The present invention provides for methods for treating animals having acute inflammatory conditions including mastitis by administering at least one p38 MAP kinase inhibitor. The present invention also provides methods for enhancing milk production and reducing milk discard in animals afflicted with acute inflammatory conditions by administering at least one, p38 MAP kinase inhibitor.

The p38 MAP kinase inhibitor compounds utilized for the present invention may be synthesized by synthetic routes that include processes analogous to those well-known in the chemical arts, particularly in light of the description contained herein or through the references cited.

The compound of Formula I is also a p38 MAP kinase inhibitor, and is useful in the treatment of inflammation, osteoarthritis, rheumatoid arthritis, cancer, reperfusion or ischemia in stroke or heart attack, autoimmune diseases, and other disorders.

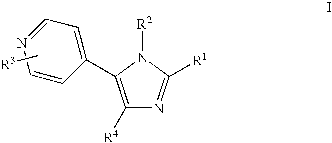

The compounds of Formula I, a prodrug of said compound or isomer, or a pharmaceutically acceptable salt of said compound, isomer or prodrug; wherein R¹ is —H;

R² is substituted and unsubstituted heterocyclic, cycloalkyl, aryl, heteroaryl: wherein heterocyclic is a 5-, 6- or 7-membered saturated, partially saturated or unsaturated ring containing from one to three heteroatoms independently selected from the group consisting of nitrogen, oxygen and sulfur; and including any bicyclic group in which any of the above heterocyclic rings is fused to a benzene ring or another heterocycle; and the nitrogen may be in the oxidized state giving the N-oxide form; and optionally substituted with R_{y};

R_{y} for each occurrence is independently -halo, —OH, —(C₁-C₆)alkyl, —(C₂-C₆)alkenyl, —(C₂-C₆)alkynyl, —O(C₁-C₆)alkyl, —O(C₂-C₆)alkenyl, —O(C₂-C₆)alkynyl, —(C₀-C₆)alkyl-NR¹³R¹⁴, —C(O)—NR¹³R¹⁴, —SO₂R¹³, —SOR¹³, —SR¹³, —NR¹³—SO₂R¹⁴, —NR¹³—C(O)—R¹⁴, —NR¹³—OR¹⁴, —SO₂—NR¹³R¹⁴, —CN, —CF₃, —C(O)(C₁-C₆)alkyl, ═O, —SO₂-phenyl, or C(O)—Ar or het-Ar;

R³ is independently —H, -halo, —OH, —(C₁-C₁₀)alkyl, OCH₃, NH₂, NHR, wherein R is aryl, heteroaryl or alkyl; and

R⁴ is substituted and unsubstituted aryl and heteroaryl;

R¹³ and R¹⁴ for each occurrence are each independently —H; —(C₁-C₆)alkyl, wherein 1 or 2 carbon atoms, other than the connecting carbon atom, may optionally be replaced with 1 or 2 heteroatoms independently selected from S, O and N and wherein each carbon atom is optionally substituted with 1, 2 or 3 halo; —(C₂-C₆)alkenyl, optionally substituted with 1, 2 or 3 halo; or —(C₂-C₆)alkynyl wherein one carbon atom, other than the connecting carbon atom and the ethynyl atoms, may optionally be replaced with one oxygen atom and wherein each carbon atom is optionally substituted with 1, 2 or 3 halo;

or R¹³ and R¹⁴ are taken together with N to which they are attached to form het.

In particular, the compound, MAPKi #1, is a species of the genus described in compound of Formula I. MAPKi #1 is the subject of W095/02591A1 and W096/21452A1 (hereby incorporated by reference in its entirety) and may be prepared as more fully described therein.

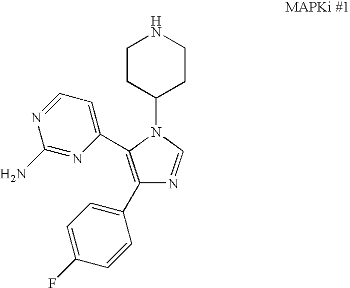

The compound of Formula II, 5-(phenylheteroaryl)-1,3-dihydro-2-benzimidazolones, is also a p38 MAP kinase inhibitor, and is useful in the treatment of inflammation, osteoarthritis, rheumatoid arthritis, cancer, reperfusion or ischemia in stroke or heart attack, autoimmune diseases, and other disorders.

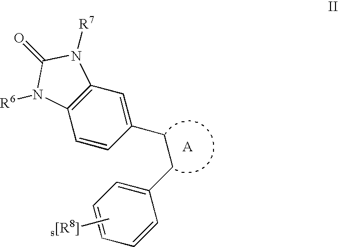

The compound of Formula II, wherein “A” is substituted or unsubstituted pyrrolyl, imidazolyl, pyrazolyl, oxazolyl, isoxazolyl, thiazolyl, or isothiazolyl; R⁶ and R⁷ are independently H or substituted or unsubstituted (cyclo)alkyl, phenyl, heteroaryl, or heterocyclyl; R⁸ is independently halo, (perhalo)alkyl, (perhalo)cycloalkyl, alkenyl, alkynyl, heterocyclyl(oxy), phenyl, OH, (perhalo)alkoxy, phenoxy, alkylthio, alkyl(amino)sulfonyl, alkylsulfamoyl, carbamoyl, acyl or carboxy; and s is 0-5 is the subject of WO 2002/072576 (hereby incorporated by reference in its entirety) and the preparation of the compound of Formula II is described therein.

The compounds of Formula II, wherein “A,” R⁶, R⁷, R⁸ and s are defined above, may be prepared, as more fully described in WO 2002/072576 (Note: the variables “A, R⁶, R⁷, R⁸” may be identified with different designations in WO 2002/072576). In particular, the compound, MAPKi #2, may be prepared as set forth below in Scheme I.

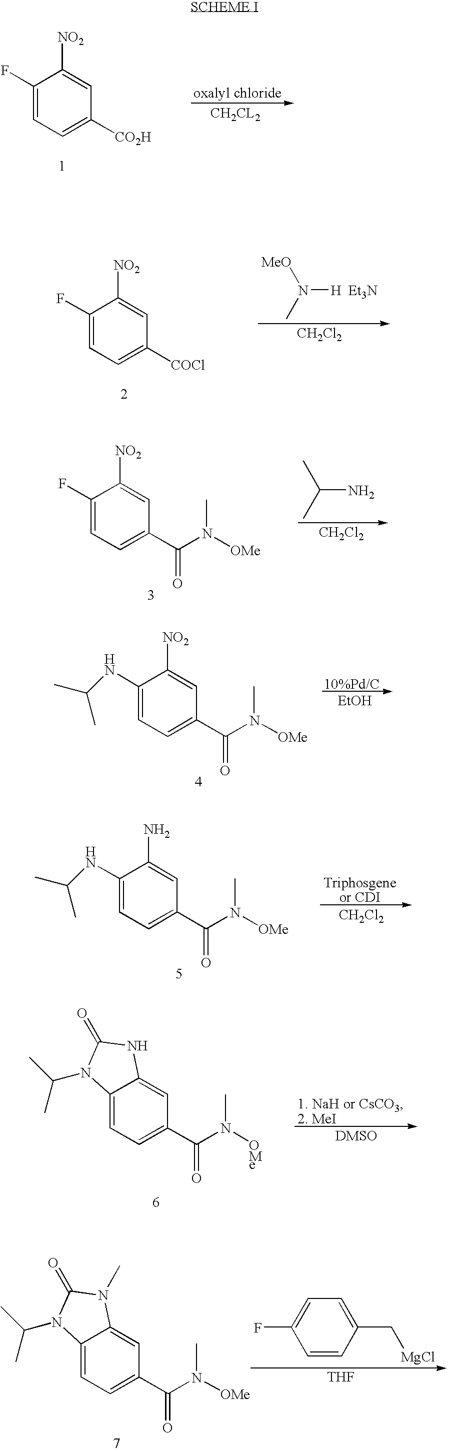

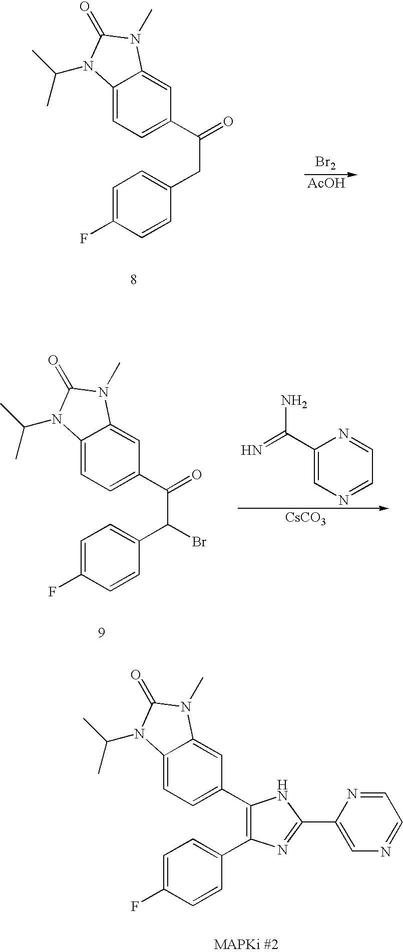

The compound, MAPKi #2, was prepared as set forth above in Scheme I. 4-Fluoro-N-methoxy-N-methyl-3-nitro-benzamide (3) was prepared by taking up 4-Fluoro-3-nitrobenzoic acid (1) (100 g, 0.54 mol) in dry methylene chloride (1 L) and 1.5 mL of DMF was added. To this solution was added oxalyl chloride (61 mL, 0.702 mol). After 1.5 hours, the solvent was removed in vacuo and the crude acid chloride (2) (yellow oil) was taken up in methylene chloride (50 mL) and slowly added to a stirring mixture of triethylamine (150.5 mL, 1.08 mol) and Wainreb amine hydrochloride (68.5 g, 0.702 mol) in methylene chloride (950 mL) at 0° C. The reaction was allowed to warm to room temperature and stirred overnight. The reaction mixture was washed with saturated sodium dihydrogen phosphate, followed by water. The organic phase was dried over anhydrous sodium sulfate and concentrated in vacuo to an orange-yellow oil. The crude oil was triturated with pantane to give 110.28 g (90%) of a yellow to off-white powder (3).

To a solution of 4-fluoro-N-methoxy-N-methyl-3-nitro-benzamide (3) (20 gm, 87.6 mmol) in methylene chloride (250 mL) was added isopropyl amine (11.4 g, 192.8 mmol) in several portions. The reaction was stirred overnight at room temperature; the reaction was determined to be complete by ¹HNMR the following morning. The reaction mixture was washed with water (2×100 mL) and the organic phase was dried over anhydrous sodium sulfate and concentrated in vacuo to afford 24.95 g (97%) of a yellow solid (4).

To a solution/slurry of 4-isopropylamino-N-methoxy-N-methyl-3-nitro-benzamide (4) (24.95 g, 93 mmol) in ethanol (500 mL) was added 10% Pd on carbon (approximately 1 g). The resulting black slurry was hydrogenated on a Parr shaker for 5 hours, after which TLC (ethyl acetate) showed complete consumption of starting material. (Also, the color of the ethanol solution goes from bright yellow to clear, indicating complete consumption of starting material.) The catalyst was removed by filtration through (celite, and the solvent was removed in vacuo to give 21.95 gm (>98%) of the substituted aniline (5) as a dark purple, viscous oil which was used without further purification.

To a solution of 3-amino-4-isopropylamino-N-methoxy-N-methyl-benzamide (5) (21.95 g, 92 mmol) in methylene chloride (300 mL) was added triphosgene (27 g, 92 mmol) in small portions (CAUTION: GAS EVOLUTION). After the addition of the triphosgene was complete, the reaction was stirred overnight at room temperature, after which time ¹HNMR and TLC showed complete reaction. The organic phase was washed three times with saturated sodium bicarbonate solution, the organic phase was dried over anhydrous sodium sulfate and concentrated in vacuo to give 24.2 g (quantitative) of a red foam (6) which was used without further purification.

An alternate cyclization to avoid the use of triphosgene can be accomplished by using carbonyl diimidazole. To a stirred solution of 3-amino-4-tert-butylamino-N-methoxy-N-methyl-benzamide (22.5 g, 89.5 mmol) in dry methylene chloride (250 mL) was added carbonyl diimidazole (16.0 g 98.5 mmol) in small portions (heat evolution). After stirring for 4 hours, ¹HNMR of an aliquot of the reaction showed no starting material. Saturated sodium bicarbonate solution was added to the reaction mixture and the organic phase was separated, washed with saturated bicarbonate solution and brine, then dried over anhydrous sodium sulfate. Concentration in vacuo afforded 25.68 g of a dark foam (6, but with alternate amine). which was used without further purification.

To a slurry of sodium hydride (720 mg of a 60% dispersion in mineral oil, 18 mmol) in dry DMSO (25 mL) was added 1-isopropyl-2-oxo-2,3-dJhydro-1H-benzoimidazole-5-carboxylic acid methoxy-methyl-amide (6). (4.2 g, 16 mmol) in small portions (CAUTION: GAS EVOLUTION). The resulting mixture was stirred for 30 minutes during which time the solution turned brown. A solution of methyl iodide (1.5 mL, 24 mmol) in DMSO (10 mL) was then added dropwise and the resulting solution stirred until TLC (ethyl acetate) showed complete reaction. The reaction was quenched with water (15 fold volume excess) and the aqueous phase was extracted with ethyl acetate (3×200 mL). The combined organic phases were washed with water and brine. The organic phase was dried over anhydrous sodium sulfate and concentrated in vacuo to afford 4.8 g (98%) of a brown oil (7).

An alternate alkylation may be accomplished by using cesium carbonate. To a stirred solution of 4-amino-3-ethoxycarbonylamino-benzoic acid methyl ester (23.6 g, 99 mmol) in DMF (330 mL, 0.3 M final concentration) was added cesium carbonate (114 g, 350 mol, 3.5 eq.). The resulting green slurry was heated at 70° C. overnight, after which time ¹HNMR of an aliquot of the reaction mixture showed complete cyclization (as the reaction proceeds, the color changes from green to brown. The reaction was then cooled to room temperature and ethyl iodide (22,7 mL, 218 mmol) was added. The reaction was stirred at room temperature for 1 hour after which time ¹HNMR of an aliquot showed complete reaction. The reaction mixture was then diluted with water (15 volumes) and the resulting aqueous layer was extracted with ethyl acetate (3×150 mL). The organics were combined, washed with 1N HCl and water. The organic phase was dried over anhydrous sodium sulfate and concentrated in vacuo to give 19.4 g of an orange solid (7, but with alternate amine) which was used without further purification.

To a stirred solution of 1-isopropyl-3-methyl-2-oxo-2,3-dihydro-1H-benzoimidazole-5-carboxylic acid methoxy-methyl-amide (7) (17 g, 61 mmol) in dry DME (700 mL) was added p-fluorobenzylmagnesium chloride (Rieke Metals, 0.25 M in Et₂O, 400 mL, 100 mmol). The reaction mixture was allowed to stir overnight, after which time ¹HNMR of on aliquot of the reaction indicated no starting material remained. The excess Grignard reagent in the reaction mixture was quenched with saturated aqueous sodium dihydrogen phosphate, and the DME was removed in vacua. The remaining aqueous phase was extracted with ethyl acetate (3×200 mL) and the combined extracts were dried over anhydrous sodium sulfate and the solvent was concentrated in vacua. Chromatography (Flash 75, gradient elution 25% ethyl acetate-hexanes to 50% ethyl acetate-hexanes) afforded 15 g of a white solid (8).

To a stirred solution of 1-isopropyl-3-methyl-5-p-fluorophenylacetyl-1,3-dihydro-benzoimidazol-2-one (8) (10.0 g. 31 mmol) in acetic acid (60 mL) was added bromine (1.63 mL, 31.6 mmol) in one portion. The reaction was allowed to stir overnight (c.a. 4 hours) after which time it was found to be complete by ¹H NMR. The reaction was concentrated in vacuo and the residue was taken up in ethyl acetate (150 mL) and washed twice with saturated sodium bicarbonate solution. The organic phase was dried with anhydrous sodium sulfate and concentrated in vacuo to afford 12.1 g of a tan solid (9) that was used without purification.

A stirred mixture of 5-(bromo-p-fluorophenyl-acetyl)-1-isopropyl-3-methyl-1,3-dihydro-benzoimidozol-2-one (8) (0.5 g, 1.23 mmol), pyrazine-2-carboxamidine hydrochloride (0.395 g, 2,49 mmol), cesium carbonate (1.22 g, 3.74 mmol) and DMF (4.0 mL) was heated to 60° C. After 1 hour, the reaction was determined to be complete by LCMS. The reaction was cooled to room temperature and diluted with water (40 mL). After stirring for 1 hour, the crude suspension was filtered and the solids were purified by flash chromatography (diethyl ether, followed by ethyl acetate to afford 40 mg of the title compound (MAPKi #2) as a light tan solid.

The compound, MAPKi #3, was prepared as described above in Scheme I and below in Scheme II, except that in Step 3, t-butyl amine is utilized, instead of isopropyl amine, to prepare the compound of Formula 4.

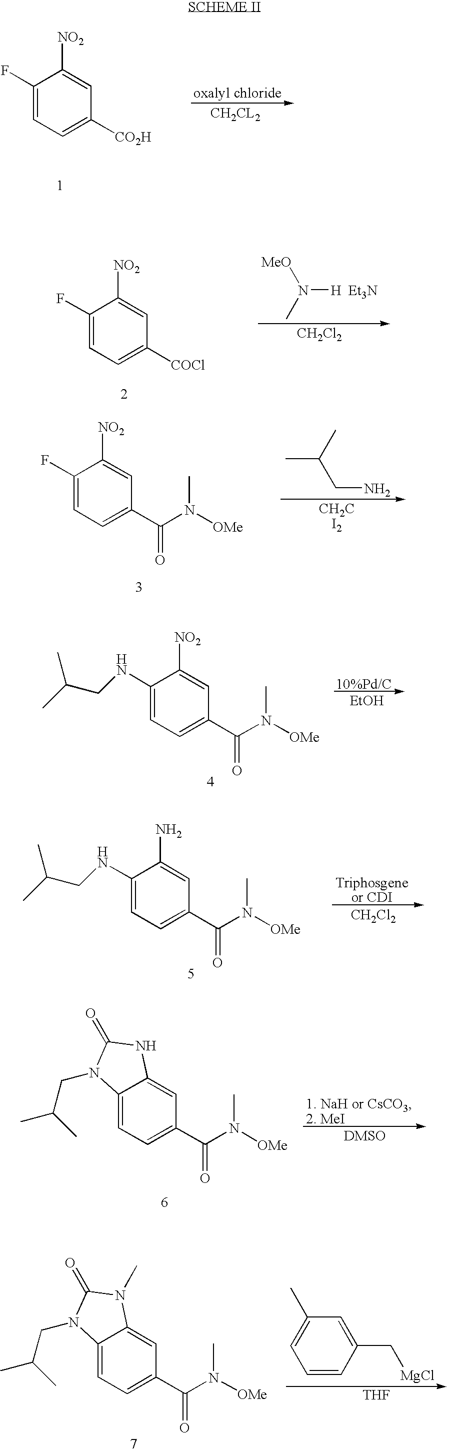

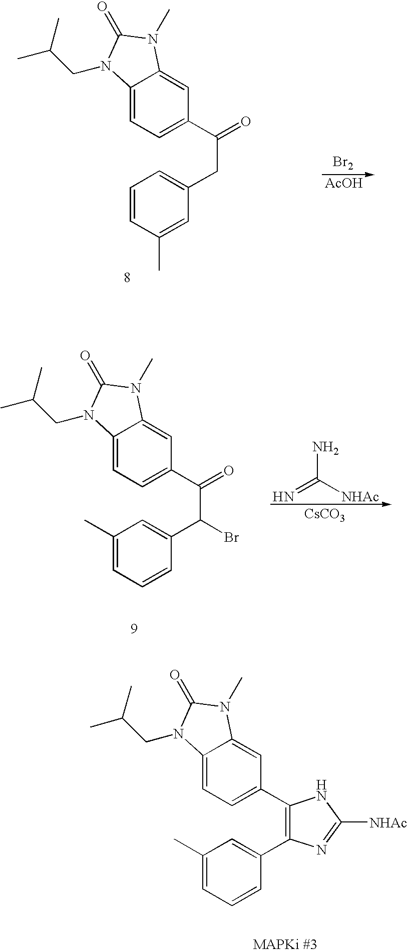

The compound of Formula III is a potent inhibitor of MAP kinases, preferably p38 kinase, and is useful in the treatment of inflammation, osteoarthritis, rheumatoid arthritis, cancer, reperfusion or ischemia in stroke or heart attack, autoimmune diseases and other disorders. The compound of Formula III, wherein “B” is a substituted or unsubstituted hetero group, such as pyrrolyl, imidazolyl, pyrazolyl, oxazolyl; R⁹ is H, alkyl, cycloalkyl, heterocycloalkyl, aryl or heteroaryl; R¹⁰ is H, alkyl, Ph, F, Cl or CN; and s is 0-5, is the subject of U.S. 2003-078432 (incorporated herein by reference in its entirety. Note the variables R⁹ and R¹⁰ are defined as different variables in US 2003-078432), and the preparation of the compound of Formula III is described therein.

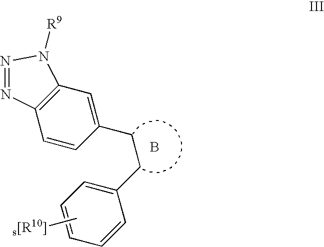

For general illustrative purposes, the reaction Scheme III depicted below provides a potential route for synthesizing the compound of Formula IIIa. For a more detailed description of the individual reaction steps, see the reference described above.

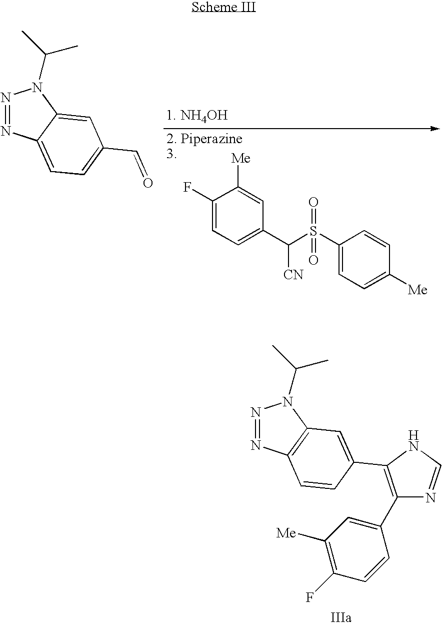

In particular, the compound of Formula III, wherein “B,’ R⁹, R¹⁰ and s are defined above, may be prepared, as set forth in Scheme III and as more fully described in U.S. 2003-078432 (Note: the designation of “B,’ R⁹, R¹⁰ is different and is described with different variables in U.S. 2003-078432) by treating a THF solution of 3-isopropyl-3H-benzotriazole-5-carbaldehydein with concentrated NH₄OH, followed by the addition of piperazine and the isocyanide compound to provide the compound of Formula IIIa.

The compounds of Formula IV, 6-(phenylheterocyclyl)-[1,2,4]triazolo[4,3-a]pyridines, are useful in the treatment of inflammation, osteoarthritis, rheumatoid arthritis, cancer, reperfusion or ischemia in stroke or heart attack, autoimmune diseases, and other disorders. The compound of Formula IV is the subject of WO 2002072579 (incorporated herein by reference in its entirety), and the preparation of the compound of Formula II is described therein.

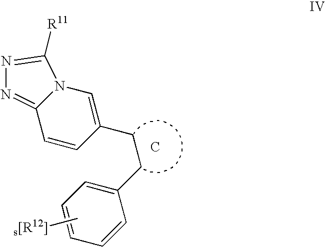

The compounds of Formula IV, wherein “C” is substituted or unsubstituted pyrrolyl, imidazolyl, pyrazolyl, oxazolyl, isoxazolyl, thiazolyl, or isothiazolyl; R¹¹ is H, alkenyl, alkynyl, or substituted or unsubstituted (cyclo)alkyl, phenyl, heteroaryl, or heterocyclyl, or amino; R¹² is halo, (cyclo)alkyl(oxy), (perhalo)alkyl, alkenyl, alkynyl, phenyl, heteroaryl(oxy), heterocyclyl(oxy), OH, (perhalo)alkoxy, phenoxy, alkylthio, alkylsulfonyl, alkylaminosulfonyl, NO₂, substituted and unsubstituted amino or carbamoyl; and s is 0-5; or pharmaceutically acceptable salts thereof, are useful in treating acute inflammation in animals.

The compounds of Formula IV, wherein “C,’ R¹¹, R¹² and s are defined above, may be prepared, as more fully described in WO 2002072579 (Note: the designation of “C,’ R¹¹, R¹² are defined with different variables in WO 2002072579). For example, the compound of Formula IVa was prepared by condensing 6-chloronicotinic acid with N,O-dimethylhydroxylamine.bul.HCl (96%). Treatment of the amide with (i-Bu)₂AlH provided the aldehyde (24%), which was then coupled with (phenyl)(p-tolylsulfonyl)methylisocyanide to afford 2-chloro-5-(4-phenyloxazol-5-yl)pyridine (71%). Conversion to the hydrazine (100%), followed by coupling with isobutyryl chloride and cyclization using POCl₃ (32%), produced the compound of Formula IVa.

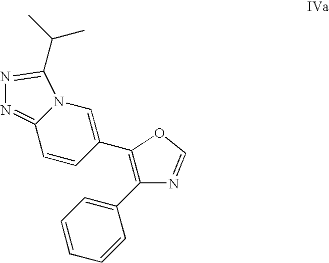

In accordance with the present invention, a subject animal suffering from an acute inflammatory condition, such as, for example, mastitis, is administered an effective amount of at least one p38 MAP kinase inhibitor and within about one to two weeks the animal produces more than twice as much milk as an infected, non-medicated animal. In a preferred embodiment the animal is a lactating cow.

Lactating animals suffering from infections caused by E. coli, for example, often produce milk which contains elevated E. coli counts and which milk is not suitable for mammalian consumption, even after processing. The present invention also provides for the reduction of milk discard in an animal suffering from an acute inflammatory condition with the administration of at least one p38 MAP kinase inhibitor. Milk discard reduction is rapid and occurs within about one week. The present invention further provides methods for the reduction in E. coli numbers in milk samples from animals treated with p38 MAP kinase inhibitors.

The p38 MAP kinase inhibitor of the present invention can be used in the treatment of an inflammatory condition in an animal, which is exacerbated or caused by excessive or unregulated cytokine production in animal cells including but not limited to monocytes and/or macrophages. Preferred p38 MAP kinase inhibitors include MAPKi #1, MAPKi #2 and MAPKi #3.

The p38 MAP kinase inhibitors of the present invention are thus capable of inhibiting the production and activity of cytokines associated with the inflammatory process such as IL-1, IL-6 and TNF and are therefore of use in therapy. IL-1, IL-6 and TNF affect a wide variety of cells and tissues and these cytokines, as well as other leukocyte-derived cytokines, are important inflammatory mediators of a wide variety of disease states and conditions. The p38 MAP kinase inhibitors of the present invention also inhibit pro-inflammatory proteins, such as COX-2, also referred to by many other names such as prostaglandin endoperoxide synthase-2 (PGHS-2). Regulation of COX-2 which is responsible for the production of proinflammatory lipid mediators also affects a wide variety of cells and tissues. The regulation of inflammatory cytokines and inflammatory proteins is thus critical for ameliorating a wide variety of diseases and conditions including, but not limited to mastitis.

Accordingly, in another aspect, the present invention provides a method of treating an animal by inhibition of the synthesis of the COX-2 enzyme comprising the administration of an effective amount of at least one p38 MAP kinase inhibitor.

The present invention also provides a method of treating cytokine-mediated acute inflammation which comprises administering an effective amount of a p38 MAP kinase inhibitor and a pharmaceutically acceptable carrier. In one embodiment the present invention provides a method of inhibiting TNF. In another embodiment the present invention provides a method of inhibiting IL-1. In still another embodiment, the present invention provides a method of inhibiting apoptotic cell death mediated through the p38 MAP kinase pathway.

In particular, p38 MAP kinase inhibitors are employed in the treatment of a disease or condition in an animal which is exacerbated by or caused by excessive or unregulated IL-1 or TNF production in animal cells including but not limited to, monocytes and/or macrophages.

There are many conditions or diseases in which excessive or unregulated cytokine production is implicated in exacerbating and/or causing disease. These include acute inflammatory disease states in animals such as mastitis, respiratory disease, retained placental membranes, metritis, pyrometra, enteritis, hepatitis, nephritis, septicemia, laminitis, frost bite, colic, displaced abomasums, endotoxemia and cecal torsion.

The p38 MAP kinase inhibitors are administered in an amount sufficient to inhibit cytokine effects and production, in particular IL-1, IL-6 or TNF, production such that cytokine production is down-regulated to normal levels, or in some case to subnormal levels, so as to ameliorate or prevent the disease state. Cytokine level measurement is accomplished by the skilled artisan using conventional means.

As used herein, the term “cytokine” refers to any secreted polypeptide that affects the functions of cells and is a molecule which modulates interactions between cells in the inflammatory response. A cytokine includes, but is not limited to, monokines and lymphokines, regardless of which cells produce them. Examples of cytokines include, but are not limited to, Intrerleukin-1, (IL-1), Tumor Necrosis Factor-alpha (TNF-α) and Tumor Necrosis Factor beta (TNF-β).

In order to employ a p38 MAP kinase inhibitor in therapy, such inhibitor will normally be formulated into a pharmaceutical composition in accordance with standard pharmaceutical practice. This invention, therefore, also relates to a pharmaceutical composition comprising an effective, non-toxic amount of at least one p38 MAP kinase inhibitor and a pharmaceutically acceptable carrier.

p38 MAP kinase inhibitors and pharmaceutical compositions incorporating such may be conveniently administered to an animal by any of the routes conveniently used for drug administration, for instance, orally, topically, parenterally or by inhalation. The p38 MAP kinase inhibitors may be administered in conventional dosage forms prepared by combining a p38 MAP kinase inhibitor with standard pharmaceutical carriers according to conventional procedures. The p38 MAP kinase inhibitor may also be administered in conventional dosages in combination with a known, second therapeutically active compound or two or more p38 MAP kinase inhibitors can be administered at once to take advantage of the synergistic properties of the p38 MAP kinase inhibitors and provide enhanced inhibition of inflammation and conditions caused thereby.

Procedures for administering conventional dosages of p38 MAP kinase inhibitors may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the animal recipient thereof.

The pharmaceutical carrier employed may be, for example, either a solid or liquid. Exemplary of solid carriers are lactose, sucrose, talc, gelatin, agar, pectin, acacia, magnesium stearate, steric acid and the like. Exemplary of liquid carriers are syrup, peanut oil, olive oil, water and the like. Similarly, the carrier or diluent may include sustained release material well known to the art, such as glyceryl monostearate or glyceryl distearate alone or with a wax.

The term “systemic administration” refers to intravenous, subcutaneous and intramuscular administration. Systemic administration is preferred. p38 MAP kinase inhibitors are preferably administered parenterally that is by intravenous, intramuscular, intramammary or subcutaneous administration. The subcutaneous and intramuscular forms of parental administration are generally preferred. Appropriate dosage forms for such administration may be prepared by conventional techniques.

For all methods of use disclosed herein for p38 MAP kinase inhibitors, the parenteral dosage regimen will preferably be from about 0.05 mg/kg to about 20 mg/kg of total body weight, preferably from about 0.1 mg/kg to 5 mg/kg, more preferably from about 0.1 mg/kg to 1 mg/kg. It will also be recognized by one of skill in the art that the optimal quantity and spacing of individual dosages of p38 MAP kinase inhibitors thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a p38 MAP kinase inhibitor given per day for a defined number of days, can be ascertained by those skilled in the art using conventional course of treatment determination tests.

### EXAMPLE

Thirty-three lactating Holstein cows were randomly allotted to 5 treatment groups blocked by milk production and days in milk. Milk and blood samples and temperature data were collected at the morning milking on day −1. One normal quarter was selected from each cow based on clinical scores and California Mastitis Test (CMT) results performed at morning milking on day −1. After the evening milking on day 41, selected quarters were infused with approximately 30 cfu of E. coli (MacDonald 487). Milk and blood samples and temperature data were collected prior to treatment at the morning milking on day 0. Cows were treated after the morning milking on day 0 according to the study design. After treatment, milk and blood samples and temperature data were collected at 11, 24, 35, 48, 72, 144, 168, 192, and 216 hours. Clinical scores of infused quarters and milk production were assessed at each milking. Milk samples were analyzed for culture (E. coli), SCC, TNF-α and PGE₂ and blood leukograms were determined.

TABLE 1

| | | | Number of |
|---|---|---|---|
| Treatment | Dose | Route | Animals |
| 1. Non-infected, Vehicle-treated* | | IV | 6 |
| 2. Infected, Vehicle-treated | | IV | 6 |
| 3. Infected, MAPKi #1 | 10 mg/kg | IV | 7 |
| 4. Infected, MAPKi #2 | 10 mg/kg | IV | 7 |
| 5. Infected, MAPKi #3 | 10 mg/kg | IV | 7 |

*Vehicle in this study was 25% N-methyl-pyrrolidone and 25% dimethylsulfoxide in polyethylene glycol of a nominal weight of 400 Daltons.

### Data Analysis

Assessment of test article efficacy was determined based upon a comparison of the treatment effect on each variable versus the challenged, non-medicated group. Data were analyzed using the MIXED procedure of PC-SAS version 6.12. The model included treatment, time and their interaction. Covariance within cows across time was modeled using the Repeated statement analysis with a spherical covariance structure to account for unequally spaced sampling times. Tests for significance (P≦0.10) were based upon the main treatment effect compared with the challenged, non-medicated group. The P value of ≦0.10 was selected based on the number of animals per treatment group and the conservative nature of the SAS procedure.

### Response to E. coli Challenge

Challenge of a single quarter of each cow with ˜30 cfu of E. coli (MacDonald 487 strain) resulted in a severe mastitis within 13-24 hours. In all, 27 of 27 quarters challenged developed clinical mastitis. Peak rectal temperature and bacterial colony count data after challenge suggest that the resulting mastitis was more severe than observed in previous studies.

### Effects of Treatment on Acute Phase Response to E. coli Induced Mastitis Body Temperature

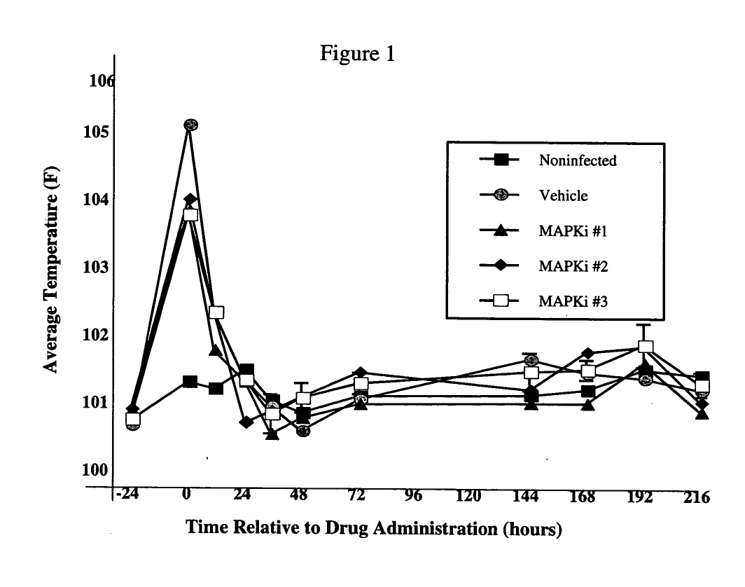

No significant differences in body temperature were observed after treatment between cows treated with any of the p38 MAP kinase inhibitor compounds and infected, non-medicated cows (FIG. 1).

### Milk Production

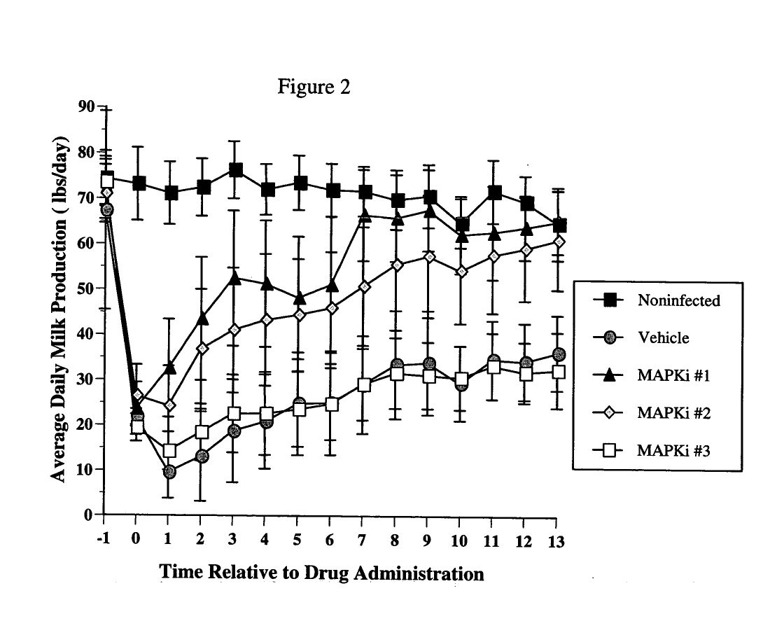

Cows treated with MAPKi #1 demonstrated significantly less milk production loss associated with mastitis following treatment compared to infected cows treated with vehicle (P=0.1, FIG. 2). Based on the mean daily milk production values from FIG. 2, a cow treated with MAPKi #1 produced more than twice as much milk as an infected, non-medicated cow during the 13 days after treatment (847 lbs vs. 365 lbs). Cows treated with MAPKi #2 also produced more milk than control cows (689 lbs vs. 365 lbs). Cows treated with MAPKi #3 produced the same amount of milk as infected, non-medicated control cows during that time period (366 lbs vs. 365 lbs).

### Clinical Score

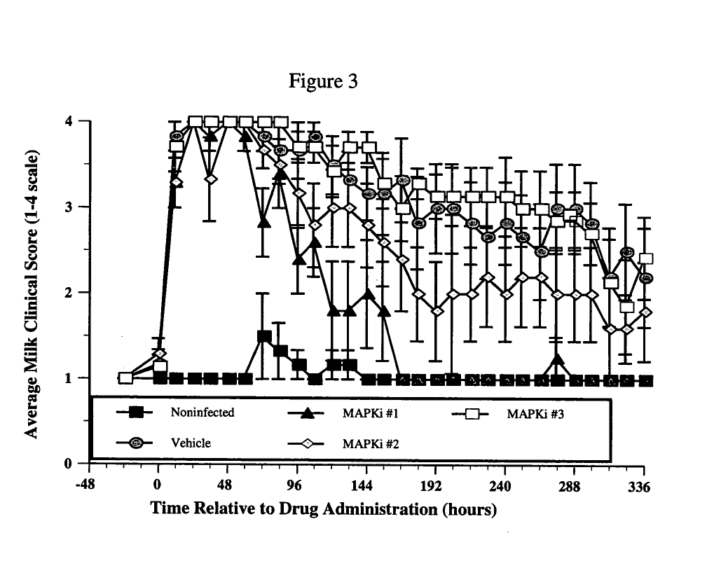

Cows treated with MAPKi #1 and MAPKi #2 had significantly improved milk clinical scores than the non-medicated controls (FIG. 3, P<0.001, P=0.006, respectively). Cows treated with MAPKi #3 had milk clinical scores similar to the infected, non-medicated controls.

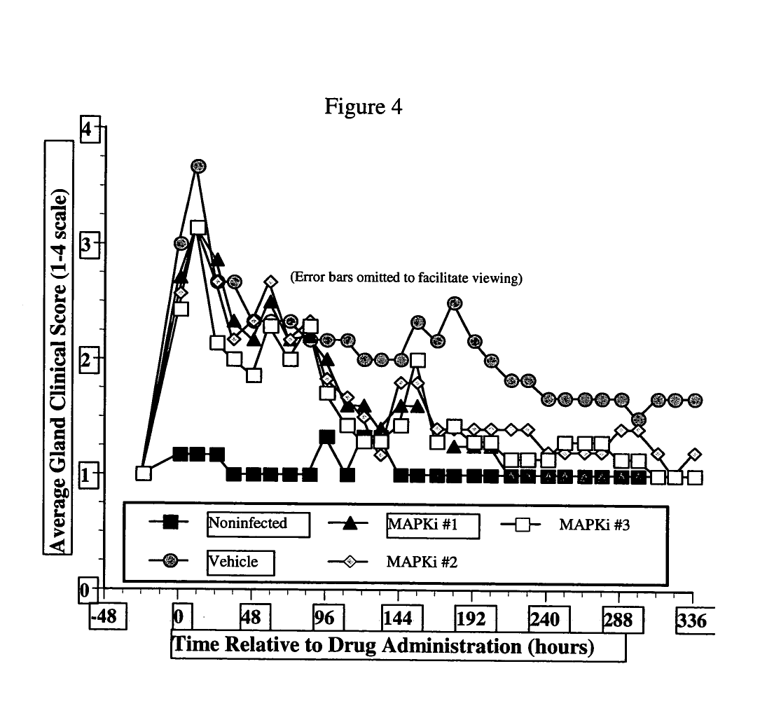

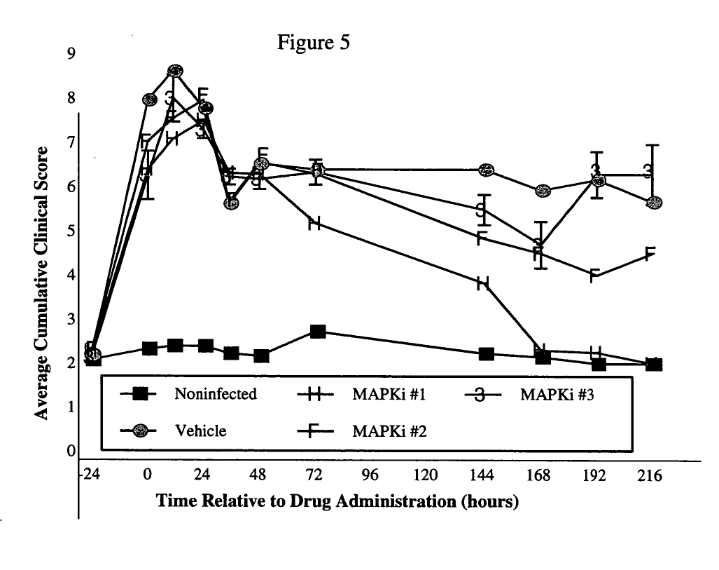

Significantly improved gland clinical scores were observed for cows treated with any of the three p38 MAP kinase inhibitor compounds (FIG. 4, P=0.0004, P=0.004, P<0.001, respectively for MAPKi #1, MAPKi #2, MAPKi #3). Cows treated with MAPKi #1 and MAPKi #2 demonstrated significant improvement in both milk and gland score. Cumulative clinical scores were also significantly lower for cows treated with MAPKi #1 and MAPKi #2 compared to controls (FIG. 5, P=0.0001 and 0.07, respectively).

### Milk Somatic Cell Count (SCC)

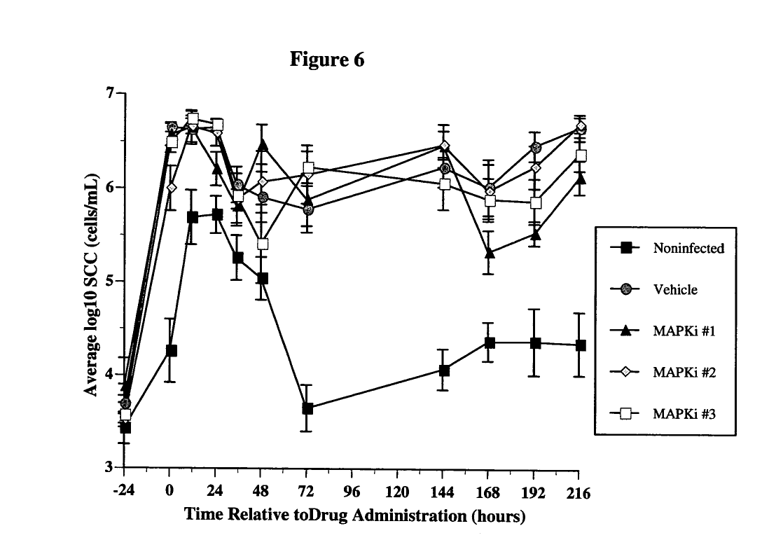

Milk SCCs were increased at 13 hours post-challenge for cows in all treatment groups (FIG. 6). Some milk samples that were grossly clotted were not analyzed for SCC, instead they were assigned the maximum value readable by the instrument of 10,000,000 cells/ml for data analysis purposes (log₁₀=7). Some normal milk samples had SCC lower than the detectable limit of the instrument (2000 cells/ml) and were assigned a value of 1000 instead of 0 for data analysis purposes. Treatment of cows with the p38 MAPKi compounds did not significantly improve SCC compared to infected, non-medicated control cows in this study. Cows treated with MAPKi #1 and MAPKi #3 did show a trend towards lower SCC, particularly after 6 days post-treatment (P=0.13, 0.18, respectively).

### Leukograms

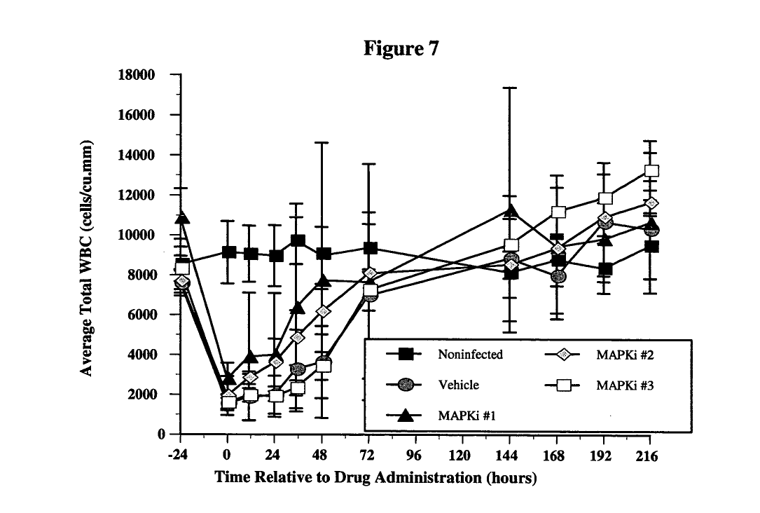

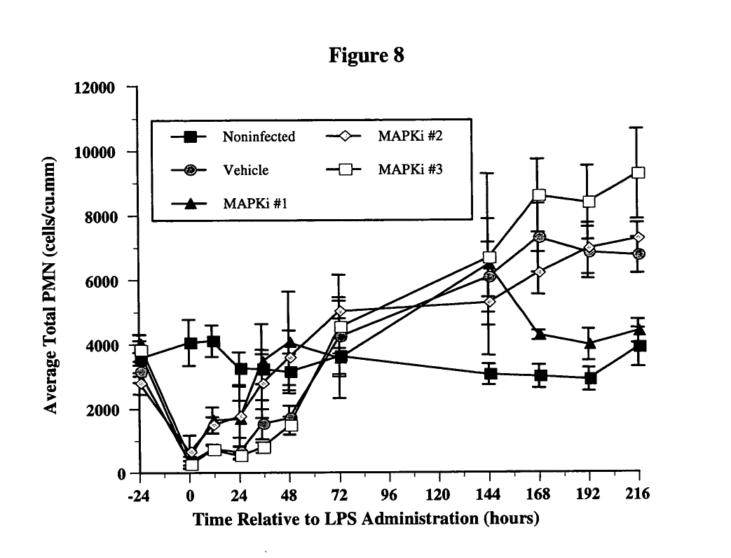

Total white blood cell and total neutrophil counts remained relatively unchanged throughout the study for non-infected, non-medicated cows (FIGS. 7 and 8). Infected, non-medicated cows demonstrated the typical biphasic response for WBCs and PMNs. Both dropped dramatically after challenge (0-72 hours) and the WBC numbers returned to near pre-challenge levels and PMN numbers reached more than double the pre-challenge levels late in the study (144-216 hours). Total WBC and PMN numbers for cows treated with MAPKi #1 or MAPKi #2 returned to pre-challenge levels significantly faster than for controls or cows treated with MAPKi #3 when data was analyzed for 0-72 hours (P=0.004, 0.06, respectively). These data suggest that soon after treatment with MAPKi #1 or MAPKi #2, fewer PMNs were recruited into the mammary gland from the peripheral blood. It is known that neutrophils contribute to the tissue pathology in the mammary gland when they lyse and release their contents to the surrounding environment. Fewer neutrophils recruited to the gland results in less tissue pathology. Less tissue pathology results in a reduction in loss of milk production and a quicker return to normal milk and glands.

### Whey PGE₂

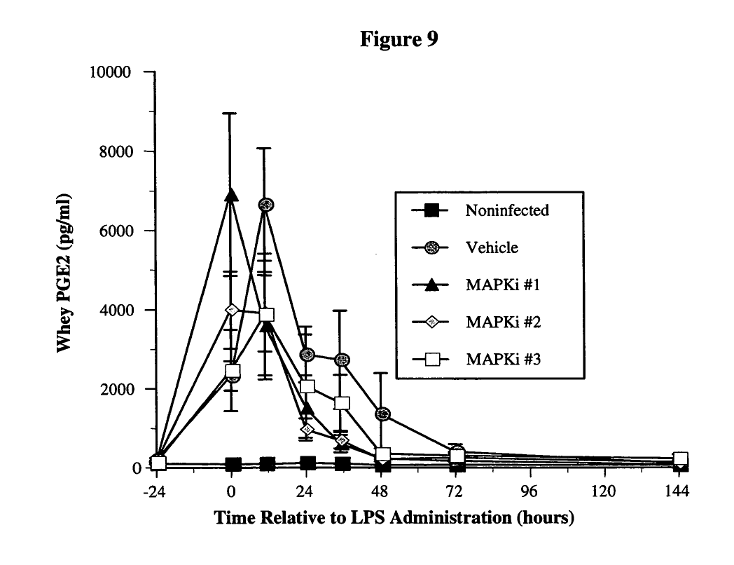

After treatment at time 0 cows treated with MAPKi #1, MAPKi #2, or MAPKi #3 all demonstrated a significant reduction in mean whey PGE₂ levels compared to infected, non-medicated control cows (FIG. 9, P=0.0038, 0.0741, 0.0934, respectively). Whey PGE₂ levels were elevated at 13 hours after E. coli challenge (time 0) and prior to treatment. After treatment, cows treated with MAPKi #1 showed a decline in whey PGE₂ levels at 11 hours, whey PGE₂ of cows treated with MAPKi #2 remained at the same level and cows treated with MAPKi #3 had whey PGE₂ levels continue to rise. Cows from all three treatment groups demonstrated declining whey PGE₂ levels 24 hours after treatment and all had returned to near baseline by 48 hours.

### Milk E. coli Colony Counts

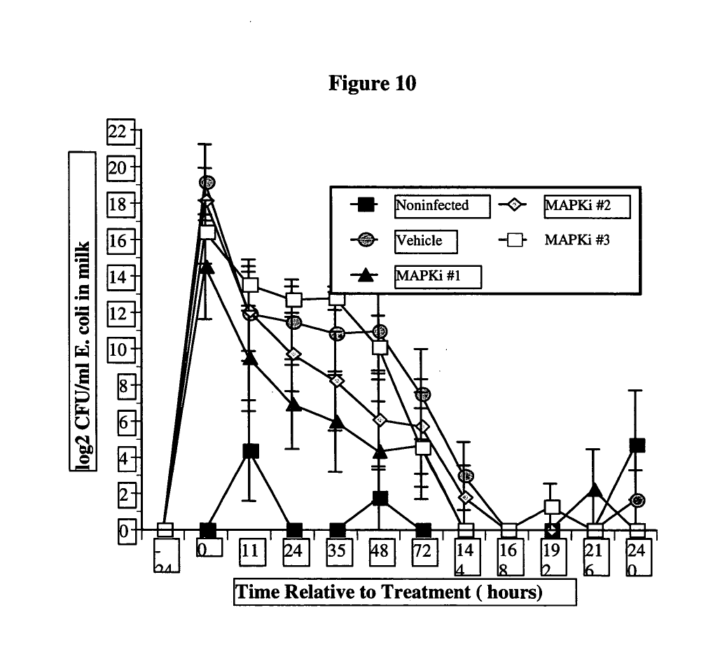

E. coli numbers in milk samples are illustrated in FIG. 10. At the time of treatment (0 hour) mean E. coli colony counts in milk ranged from 14.5-19.1 log2 cfu/ml for challenged groups. After treatment (11-168 hours) mean E. coli colony counts were significantly decreased for cows treated with MAPKi #1 compared to infected, non-medicated controls (P=0.007). Cows treated with MAPKi #2 also showed a decline in milk E. coli numbers compared to controls but the difference was not significant (P=0.15). Cows treated with MAPKi #3 did not show a decline in milk E. coli numbers compared to controls. It is not believed that these compounds are directly antibacterial in nature but the p38 MAPK enzyme itself is involved in a number of cell processes, some of which may influence growth and/or survival of bacteria in the milk and mammary gland. Natural antibacterial peptides produced by bovine neutrophils (defensins) may be blocked or inhibited by the p38 MAPK enzyme. Inhibition of the enzyme by these compounds may allow natural release of these peptides and enhance bacterial killing by neutrophils. Another possible explanation is the p38 MAPK enzyme system contributes to activating apoptosis of neutrophils, by inhibiting p38 MAPK neutrophil apoptosis may be delayed and prolong the ability of neutrophils to fight infection.

Challenge of cows with E. coli resulted in a 100% incidence of clinical mastitis. Significant improvement in the acute phase response (less milk production loss, improved milk clinical score, gland clinical score, cumulative clinical score, total leukocyte count, whey PGE₂ and milk E. coli count) was observed for cows treated with MAPKi #1 compared to infected, non-medicated controls. Significant reductions in milk, gland, and cumulative clinical scores and reduced milk E. coli counts were observed for cows treated with MAPKi #2 compared to control cows. Cows treated with MAPKi #3 showed significantly reduced whey PGE₂ and improved gland clinical scores but no trend for improved milk production or milk scores compared to infected controls.

1. A method of treating an inflammatory disease or enhancing the recovery from acute inflammatory disease in an animal in need thereof which comprises administering to said animal an effective amount of one or more p38 MAP kinase inhibitors.

2. A method for the enhancement of milk production or reduction of milk loss in an animal suffering from an acute inflammatory disease which comprises the administration to said mammal of an effective amount of one or more p38 MAP kinase inhibitors.

3. A method of inhibiting the synthesis and activity of the COX-2 enzyme, TNF or IL-1 in an animal comprising the administration of an effective amount of one or more p38 MAP kinase inhibitors.

4. A method of inhibiting apoptotic cell death in an animal comprising the administration of an effective amount of one or more p38 MAP kinase inhibitors.

5. The method of treating inflammatory disease, enhancing the recovery from acute inflammatory disease, enhancing milk production or reduction of milk loss in an animal suffering from an acute inflammatory disease, or inhibiting the synthesis and activity of the COX-2 enzyme, TNF or IL-1 in an animal with a p38 MAP kinase inhibitor wherein the p38 MAP kinase inhibitor is selected from
(i) the compound of Formula I, 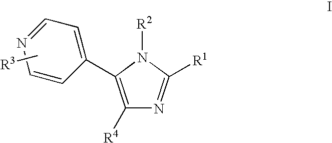
wherein R¹ is —H;
R² is substituted and unsubstituted heterocyclic, cycloalkyl, aryl, heteroaryl: wherein heterocyclic is a 5-, 6- or 7-membered saturated, partially saturated or unsaturated ring containing from one to three heteroatoms independently selected from the group consisting of nitrogen, oxygen and sulfur; and including any bicyclic group in which any of the above heterocyclic rings is fused to a benzene ring or another heterocycle; and the nitrogen may be in the oxidized state giving the N-oxide form; and optionally substituted with R_{y};
R_{y} for each occurrence is independently -halo, —OH, —(C₁-C₆)alkyl, —(C₂-C₆)alkenyl, —(C₂-C₆)alkynyl, —O(C₁-C₆)alkyl, —O(C₂-C₆)alkenyl, —O(C₂-C₆)alkynyl, —(C₀-C₆)alkyl-NR¹³R¹⁴, —C(O)—NR¹³R¹⁴, —SO₂R¹³, —SOR¹³, —SR¹³, —NR¹³—SO₂R¹⁴, —NR¹³—C(O)—R¹⁴, —NR¹³—OR¹⁴, —SO₂—NR¹³R¹⁴, —CN, —CF₃, —C(O)(C₁-C₆)alkyl, ═O, —SO₂-phenyl, or C(O)—Ar or het-Ar;
R³ is independently —H, -halo, —OH, —(C₁-C₁₀)alkyl, OCH₃, NH₂, NHR, wherein R is Aryl, heteroaryl or alkyl; and
R⁴ is substituted and unsubstituted aryl and heteroaryl;
R¹³ and R¹⁴ for each occurrence are each independently —H; —(C₁-C₆)alkyl, wherein 1 or 2 carbon atoms, other than the connecting carbon atom, may optionally be replaced with 1 or 2 heteroatoms independently selected from S, O and N and wherein each carbon atom is optionally substituted with 1, 2 or 3 halo; —(C₂-C₆)alkenyl, optionally substituted with 1, 2 or 3 halo; or —(C₂-C₆)alkynyl wherein 1 carbon atom, other than the connecting carbon atom and the ethynyl atoms, may optionally be replaced with 1 oxygen atom and wherein each carbon atom is optionally substituted with 1, 2 or 3 halo;
or R¹³ and R¹⁴ are taken together with N to which they are attached to form het;
(ii) the compound of Formula II, 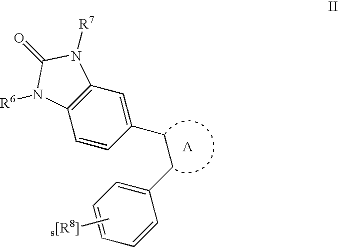
wherein “A” is substituted or unsubstituted pyrrolyl, imidazolyl, pyrazolyl, oxazolyl, isoxazolyl, thiazolyl, or isothiazolyl;
R⁶ and R⁷ are independently H or substituted or unsubstituted (cyclo)alkyl, phenyl, heteroaryl, or heterocyclyl;
R⁸is independently halo, (perhalo)alkyl, (perhalo)cycloalkyl, alkenyl, alkynyl, heterocyclyl(oxy), phenyl, OH, (perhalo)alkoxy, phenoxy, alkylthio, alkyl(amino)sulfonyl, alkylsulfamoyl, carbamoyl, acyl or carboxy; and
s is 0-5;
(iii) the compound of Formula III 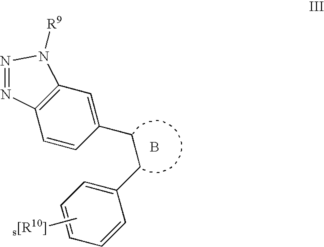
wherein “B” is a substituted or unsubstituted hetero group, pyrrolyl, imidazolyl, pyrazolyl or oxazolyl;
R⁹ is H, alkyl, cycloalkyl, heterocycloalkyl, aryl or heteroaryl;
R¹⁰ is H, alkyl, phenyl, F, Cl or CN; and
s is 0-5; or
(iv) the compound of Formula IV, 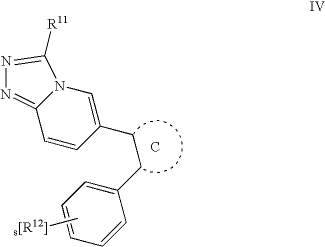
wherein “C” is substituted or unsubstituted pyrrolyl, imidazolyl, pyrazolyl, oxazolyl, isoxazolyl, thiazolyl, or isothiazolyl;
R¹¹ is H, alkenyl, alkynyl, or substituted or unsubstituted (cyclo)alkyl, phenyl, heteroaryl, or heterocyclyl, or amino;
R¹² is halo, (cyclo)alkyl(oxy), (perhalo)alkyl, alkenyl, alkynyl, phenyl, heteroaryl(oxy), heterocyclyl(oxy), OH, (perhalo)alkoxy, phenoxy, alkylthio, alkylsulfonyl, alkylaminosulfonyl, NO₂, substituted and unsubstituted amino or carbamoyl; and
s is 0-5.

6. The method of claim 5 wherein the p38 MAP kinase inhibitor is
(i) a compound MAPKi #1 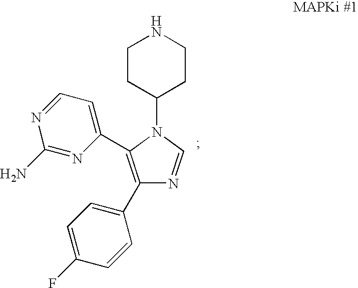
(ii) a compound MAPKi #2 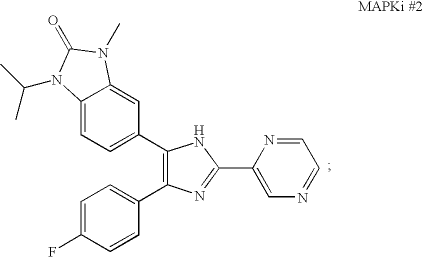
(iii) a compound MAPKi #3 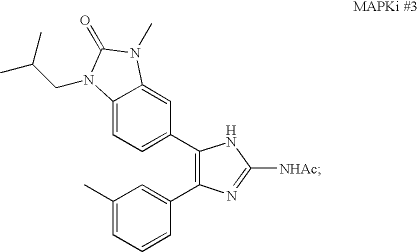
(iv) a compound of Formula IIIa 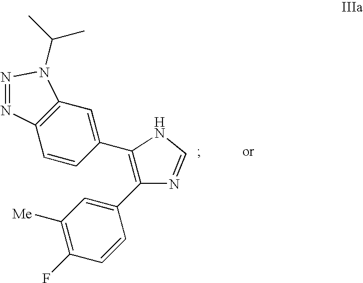
(v) a compound of Formula IVa

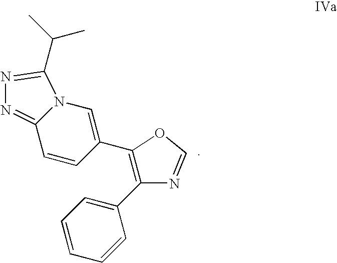

7. The method of claim 5 wherein the inflammatory disease is selected from the group consisting of mastitis, respiratory disease, replaced placenta membranes, metritis, pyometra, enteritis, hepatitis, nephritis, septicemia, endotoxemia, laminitis, frostbite and obstructive bowel problems.

8. The method of claim 5 wherein the obstructive bowel problems are selected from the group consisting of colic, displaced abomasums, and cecal torsion.

9. The method of claim 5 wherein the inflammatory disease is mastitis and the animal is a cow.

10. The method of claim 5 further comprising a pharmaceutically acceptable carrier.

11. The use of the compounds of claim 5 in the manufacture of a medicament for the therapeutic and/or prophylactic treatment of the diseases of claims 5.

12. The use of the compounds of claim 5 in the preparation of an inhibitor of one or more p38 MAP kinase inhibitors for the enhancement of milk production or reduction of milk loss or discard in an animal.

13. The use of the compounds of claim 5 in the preparation of an inhibitor of a COX-2 enzyme, TNF, IL-1 or the inhibiting of apoptotic cell death for treating or preventing the reduction of milk loss in an animal suffering from an acute inflammatory disease.

14. A process for the manufacture of a medicament for use in the treatment of inflammatory disease characterized by the use of the compounds of claim 5.

15. The use of the compounds of claim 5 in the manufacture an inhibitor of a COX-2 enzyme, TNF, IL-1 or the inhibiting of apoptotic cell death, in a package together with instructions for its use in the treatment of of inflammatory disease or reduced milk production in animals.
